(19) United States
(12) Patent Application Publication
(10) Pub. No.: US 2008/0208141 A1
(43) Pub. Date: Aug. 28, 2008

(54) Plastic Bottle for Oxaliplatin Solution

(76) Inventors: Michaela Roth, Reichersbeuern (DE); Katrin Wulfing, Munich (DE)

Correspondence Address: LICATA & TYRRELL P.C., 66 E. MAIN STREET, MARLTON, NJ, 08053, US

(21) Appl. No.: 11/813,025

(22) PCT Filed: Dec. 28, 2005
(86) PCT No.: PCT/EP2005/014098

§ 371 Date: Jul. 10, 2007

(30) Foreign Application Priority Data
Dec. 29, 2004 (DE) 102004063764.4

(51) Int. Cl. A61M 5/28; A61B 19/02

(52) U.S. Cl. 604/199; 206/438; 206/365

(57) ABSTRACT

The invention relates to a sealed plastics bottle containing oxaliplatin solution, to a kit comprising such a bottle, and to a preparation process. The plastics bottle is made, for example, of cycloolefin copolymer.

The invention relates to plastics bottles for solutions containing oxaliplatin for parenteral administration.

Oxaliplatin [cis-oxalato-(trans-1,2-diaminocyclohexane)-platinum(II)], also known as L-OHP, is one of the third generation platinum complexes. Oxaliplatin is a cytostatic and is used for treating carcinomas of the ovaries, respiratory tract, liver, breast and testicles or non-Hodgkin's lymphomas. It is used especially for the treatment of colorectal carcinoma with metastasisation.

Oxaliplatin is obtainable as a lyophilisate which is converted into a solution shortly before use. The oxaliplatin-containing solution is generally used as an infusion.

A lyophilisate has the following disadvantages:
the lyophilisation process is a relatively complicated and expensive procedure;
a lyophilisate requires an additional preparation step prior to administration, that is to say reconstitution with a solvent;
the reconstitution of the lyophilisate increases the risk of microbial contamination;
with a lyophilisate there is a risk that, on being reconstituted, the product is not fully dissolved and so particles remain that are not allowed for injection or infusion.

The following oxaliplatin formulations are described in the literature:

EP 0 774 963 B1 discloses a stable oxaliplatin solution for parenteral administration having a content of from 1 to 5 mg/ml of oxaliplatin and a pH of from 4.5 to 6. The solution is stored in a bottle made of neutral glass (paragraph number 0015).

EP 0 943 331 B1 describes a stable oxaliplatin solution containing oxalic acid or an oxalic acid salt as buffer. The solution can be introduced into an ampoule, a glass vial (page 8, line 10), an infusion pouch or a syringe. A disadvantage of that formulation is a certain toxicity of the oxalic acid.

WO 03/047 587 discloses a stable oxaliplatin solution in suitable containers (page 12, line 28) containing lactic acid or a lactic acid salt as buffer.

US 2003/0 109 515 A1 describes a stable oxaliplatin solution in suitable containers (paragraph number [0060]) containing malonic acid or a malonic acid salt as buffer.

EP 1 207 875 B1 discloses a stable parenteral solution having a concentration of at least 7 mg/ml of oxaliplatin in a solvent which comprises hydroxy compounds selected from the group 1,2-propanediol, glycerol, maltitol, saccharose and inositol. As containers there can be used multiple-dose bottles (claim 6), syringes, ampoules or infusion pouches.

WO 02/47 725 describes a stable parenteral solution having a concentration of at least 7 mg/ml of oxaliplatin, which solution has been subjected to a heat treatment at a temperature of less than 110° C. Multiple-dose bottles can be used as containers (page 4, line 5).

EP 1 121 117 B1 describes an infusion pouch with a “ready-to-use” solution containing oxaliplatin. As the material which is in direct contact with the oxaliplatin solution, polypropylene is particularly suitable. Infusion pouches have the disadvantage that they can burst under pressure.

WO 02/069 959 describes a glass bottle for an aqueous oxaliplatin solution which has a surface area to volume ratio of less than 0.26.

In accordance with the safety regulations for pharmaceutical preparations, a formulation containing oxaliplatin may not exceed a certain degree of decomposition during storage.

An overview of the relevant prior art is given in the following Table.

| Document | Priority | Bottle | Pouch |
|---|---|---|---|
| EP 0 774 963 B1 | 08 Aug. 1994 | glass flacon [0015] | |
| EP 0 943 331 B1 | 25 Feb. 1998 | glass vial [0054] | infusion bag (pouch) |
| | | | [0032] |
| EP 1 121 117 B1 | 14 Oct. 1998 | | plastics bag |
| EP 1 207 875 B1 | 30 Aug. 1999 | flacon [0024] | poche [0023] |
| WO 02/47 725 | 12 Dec. 2000 | flacon page 4, line 5 | |
| WO 02/069 959 | 02 Mar. 2001 | flacon en verre | |
| | | Claim 1 | |
| US 2003/0 109 515 | 06 Dec. 2001 | container (bottle?) | container (pouch?) |
| | | [0049], [0060] | [0049], [0060] |
| WO 03/04 587 | 06 Dec. 2001 | container (bottle?) | container (pouch?) |
| | | page 12, line 28 | page 12, line 28 |

The aim of the invention is to provide a container for oxaliplatin-containing solutions in which oxaliplatin is stable over a relatively long period. It should be economical to produce.

Surprisingly, it has been found that plastics bottles are especially suitable for storing and handling oxaliplatin solutions. That better suitability is here attributed to the lower degree of decomposition reactions of oxaliplatin solutions in a plastics bottle in comparison with a glass vessel. In a glass bottle, stronger interactions occur between the surface of the glass and the solution, the release of ions from the glass accelerating the chemical breakdown of oxaliplatin. For example, oxaliplatin solutions decompose inter alia to form oxalic acid, to form diaquo-diaminocyclohexane-platinum, the dimer thereof, and platinum(IV) complexes.

Plastics bottles are also unbreakable. As a result, the doctor, the pharmacist and the patient are protected from contamination by oxaliplatin. Unlike glass bottles, plastics bottles require no additional packaging for transport in order to avoid breakage. In addition, plastics bottles are considerably lighter than glass bottles, thus providing a saving in transport costs.

Surprisingly, it has also been found in particular that plastics bottles of cycloolefin copolymer, even when autoclaved with or without oxaliplatin solution, as is known, for example, for polypropylene from EP 1 121 117 B1 (paragraph number [0024]), release neither metal catalysts or metal nor auxiliaries of the preparation process to an extent that the stability of the oxaliplatin solutions is impaired.

The material used for the plastics bottles can be polyethylene, polypropylene, polyvinyl chloride, polycarbonate, cycloolefin copolymer (COC) or mixtures thereof. The cycloolefin copolymers are copolymers of ethylene and cyclic olefins. Suitable monomers are unsubstituted or substituted ethylenes. The cyclic olefin monomers are derived especially from dicyclopentadiene and can likewise be in unsubstituted or substituted form. The cycloolefin copolymers can be used in admixture with polypropylene, polyvinyl chloride or polyvinylidene chloride. Preference is given to the use of high-purity cycloolefin copolymers of substituted ethylene and substituted norbornene. They are available from Ticona under the trade name Topas®. They are distinguished by high breaking strength, transparency and resistance to heat, radiation and chemicals. They should be free of ions and heavy metals. They can be sterilised by means of autoclaving, ethylene oxide, gamma radiation or electron radiation. For example, Topas 8007, 6013 and 6015 exhibit lower permeability to water vapour and oxygen than polypropylene.

The plastics bottles according to the invention can be injection bottles (=vial), screw-closure bottles or ampoules.

The plastics bottles can have a cylindrical shape or have a rectangular base. Injection bottles and screw-closure bottles can contain a volume of from 1 to 1000 ml. The volume of the injection bottles is preferably from 2 to 100 ml. Ampoules can contain a volume of from 1 to 20 ml.

The plastics bottles can be colourless or coloured.

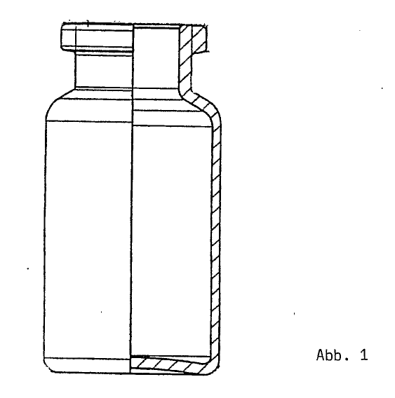

FIG. 1 shows a plastics bottle according to the invention which can be used as an injection bottle.

The plastics injection bottles can be used as single-dose or multiple-dose containers.

The plastics injection bottles can be closed with rubber stoppers. Suitable materials for the rubber stoppers are chlorobutyl or bromobutyl rubber stoppers. The stopper can be provided with a crimped cap of a lightweight metal, for example of aluminium.

The screw-closure bottles can be closed with a screw closure made, for example, of aluminium.

The term “oxaliplatin” includes cis-oxalato-(trans-l-1,2-diaminocyclohexane)-platinum (II), its optical isomer cis-oxalato-(trans-d-1,2-diaminocyclohexane)-platinum (II) and racemic mixtures thereof.

Oxaliplatin can be administered in a dose of from 10 mg/m² body surface area to 250 mg/m². The preferred dose is from 30 to 180 mg/m².

Oxaliplatin can be used in the form of aqueous solutions. Suitable solvents, in addition to water for injection purposes, are sugar solutions containing, for example, lactose, dextrose, glucose, sucrose, mannose, mannitol and/or cyclodextrins. Aqueous mixtures containing ethanol, glycerol and/or polyalkylene glycols (e.g. polyethylene glycol, polypropylene glycol, polybutylene glycol) can likewise be used.

Oxaliplatin can be used in a concentration of from 1 to 15 mg/ml, preferably from 4 to 6 mg/ml. The oxaliplatin-containing solutions according to the invention are preferably concentrates containing from 4 to 6 mg/ml.

The pH value of the oxaliplatin solution can be in the range of from 2 to 6, especially from 3 to 4.

The pH value of the solution can be adjusted with acidic organic or inorganic compounds. Suitable organic acids are, for example, citric acid, succinic acid and ascorbic acid. Examples of inorganic acids that can be used are sulfuric acid and nitric acid.

An oxaliplatin solution in a plastics bottle can be used parenterally, for example as an injection or infusion. The formulation is preferably administered intravenously. The oxaliplatin solution can be in the form of a finished solution or in the form of a concentrate. When an oxaliplatin concentrate is used, the concentrate is diluted with a carrier solution prior to administration as an injection or infusion. Suitable carrier solutions are water for injection purposes and also sugar solutions containing, for example, lactose, dextrose, glucose, sucrose, mannose and/or mannitol. Preference is given to the use of a 5% glucose solution.

An oxaliplatin solution in a plastics ampoule is preferably used as an injection.

An oxaliplatin solution in an injection bottle made of plastics is preferably used for infusion.

Preference is given to the use of an oxaliplatin-containing concentrate in a plastics injection bottle which is diluted prior to administration as an infusion.

An intravenous infusion containing oxaliplatin can be given for up to 5 days. Preference is given to a dose of from 85 to 130 mg/m² body surface area over a period of from 2 to 6 hours.

The oxaliplatin solution can be prepared by the following procedure:
dissolution of oxaliplatin in a solvent, preferably water for injection purposes
optionally, adjustment of the pH value with an acid
sterilisation of the solution
introduction of the solution into a plastics bottle
closure of the plastics bottle
   a) with a rubber stopper and crimped cap in the case of an injection bottle
   b) with a screw closure in the case of a screw-closure bottle
   c) by melt-sealing in the case of an ampoule.

A suitable material for the rubber stoppers is chlorobutyl or bromobutyl rubber, which may also have been siliconised. The rubber stoppers can be autoclaved individually and used for closing the autoclaved bottles containing the sterile solution. It is often the case that a full bottle closed with a rubber stopper is autoclaved, the rubber stopper optionally having been autoclaved beforehand.

The procedure can be carried out with or without use of an inert atmosphere. The procedure is preferably carried out under an inert atmosphere, for example under nitrogen.

The sterilisation of the solution can be effected by means of sterile-filtration or heat sterilisation. Heat sterilisation (=autoclaving) can be carried out at a temperature of at least 121° C., at a pressure of at least 2 bar for a period of at least 15 min.

The invention is explained in greater detail by the following Examples, which do not, however, limit the scope of the invention.

### EXAMPLE 1

### Composition of the Oxaliplatin Concentrate:

| Oxaliplatin | | | Material of the |
|---|---|---|---|
| concentration | Acid | pH value | plastics vial |
| 5 mg/ml | Citric acid | 3.5 | Polycarbonate |

### Preparation Procedure:

Oxaliplatin is combined with a portion of water for injection purposes and stirred until the active ingredient has completely dissolved. The pH value is then adjusted with citric acid. Water for injection purposes is then introduced to make up to the final volume of 1 ml. The solution is sterile-filtered and then introduced into plastics vials made of polycarbonate. The polycarbonates are sealed with rubber stoppers and crimped caps.

### EXAMPLE 2

### Composition of the Oxaliplatin Concentrate:

| Oxaliplatin | | | Material of the |
|---|---|---|---|
| concentration | Acid | pH value | plastics vial |
| 6 mg/ml | Sulfuric acid | 3.3 | Cycloolefin |
| | | | copolymer |

### Preparation Procedure:

Oxaliplatin is combined with a portion of water for injection purposes and stirred until the active ingredient has completely dissolved. The pH value is then adjusted with sulfuric acid. Water for injection purposes is then introduced to make up to the final volume of 1 ml. The solution is sterile-filtered and then introduced into plastics vials made of cycloolefin copolymer. They are sealed with rubber stoppers and crimped caps.

### EXAMPLE 3

Composition of the Solution Containing Oxaliplatin:

| Contents | Amount | |
|---|---|---|
| Oxaliplatin | 4 | mg |
| Water for injection | 1 | ml |
| purposes | | |

### Preparation Procedure:

Oxaliplatin is combined with water for injection purposes and stirred until the active ingredient has completely dissolved. The solution is sterile-filtered and then introduced into plastics vials made of cycloolefin copolymer. They are sealed with rubber stoppers and crimped caps.

### EXAMPLE 4

50 mg of oxaliplatin are combined with a portion of water for injection purposes and stirred until the active ingredient has completely dissolved. The pH value is then adjusted to pH=3.5 with citric acid. Water for injection purposes is then introduced to make up to the final volume of 10 ml. The solution is introduced into vials made of cycloolefin copolymer. They are sealed with rubber stoppers and crimped caps and then autoclaved at at least 121° C. and about 2 bar for more than 15 min.

After autoclaving, no decomposition of the platinum-containing compound is observed, as the following data show.

| Storage conditions | Before autoclaving | After autoclaving |
|---|---|---|
| Appearance | clear, colourless | clear, colourless |
| Sum of impurities | 0.14% | 0.17% |

1-18. (canceled)

19. A sealed plastic bottle containing an oxaliplatin solution.

20. The sealed plastic bottle of claim 19, wherein said bottle is made of polyethylene, polypropylene, polyvinyl chloride, polyvinylidene chloride, polycarbonate, cycloolefin copolymer, or a mixture thereof.

21. The sealed plastic bottle of claim 20, wherein said cycloolefin copolymer comprises ethylene and cyclic olefin.

22. The sealed plastic bottle of claim 21, wherein the ethylene is unsubstituted or substituted; and the cyclic olefin is unsubstituted or substituted, or is dicyclopentadiene or a dicyclopentadiene derivative.

23. The sealed plastic bottle of claim 22, wherein the ethylene is substituted and the cyclic olefin is substituted norbornene.

24. The sealed plastic bottle of claim 19, wherein said bottle is an injection bottle, screw-closure bottle, or ampoule.

25. The sealed plastic bottle of claim 24, wherein the bottle is closed with a rubber stopper and crimp cap, a screw closure, or is melt-sealed.

26. The sealed plastic bottle of claim 24, wherein the injection bottle is a single-dose or multiple-dose container.

27. The sealed plastic bottle of claim 19, wherein the oxaliplatin solution is water-based.

28. The sealed plastic bottle of claim 19, wherein the oxaliplatin solution is a concentrate.

29. The sealed plastic bottle of claim 27, wherein the water-based oxaliplatin solution has a pH value of from 2 to 6.

30. The sealed plastic bottle of claim 27, wherein the water-based oxaliplatin solution has a pH value of from 3 to 4.

31. The sealed plastic bottle of claim 29 or 30, wherein the pH value has been adjusted with sulfuric acid, nitric acid, citric acid, succinic acid, ascorbic acid or a mixture thereof.

32. The sealed plastic bottle of claim 19, wherein the oxaliplatin solution is maintained under an inert gas atmosphere.

33. A kit for infusion comprising a sealed plastic injection bottle containing an oxaliplatin solution.

34. The kit of claim 33, further comprising a container containing a carrier solution in an amount which is matched to the oxaliplatin solution in a pre-determined dilution ratio.

35. The kit of claim 33, further comprising a syringe.

36. The kit of claim 33, further comprising a tube with openings for pressure equalization.

37. The kit of claim 33, further comprising a vein indwelling catheter.

38. A method for producing a container containing an oxaliplatin solution comprising introducing an oxaliplatin solution into a container made of polyethylene, polypropylene, polyvinyl chloride, polyvinylidene chloride, polycarbonate, cycloolefin copolymer, or a mixture thereof; and sealing the container, thereby producing a container containing an oxaliplatin solution.

39. The method of claim 38, wherein the container and oxaliplatin solution are sterilized before sealing the container.

40. The method of claim 38, wherein the container and oxaliplatin solution are sterilized after sealing the container.

41. The method of claim 38, wherein the oxaliplatin solution is introduced into the container under an inert gas atmosphere.
